(19) United States
(12) Patent Application Publication
(10) Pub. No.: US 2013/0281758 A1
(43) Pub. Date: Oct. 24, 2013

(54) Treatment of refractory neuropsychiatric disorders using targeted radiation

(71) Applicants: Hugh Brent Solvason, Stanford, CA (US); John R. Adler, Stanford, CA (US)

(72) Inventors: Hugh Brent Solvason, Stanford, CA (US); John R. Adler, Stanford, CA (US)

(73) Assignee: The Board of Trustees of the Leland Stanford Junior University, Palo Alto, CA (US)

(21) Appl. No.: 13/865,903

(22) Filed: Apr. 18, 2013

Related U.S. Application Data
(60) Provisional application No. 61/635,033, filed on Apr. 18, 2012.

(51) Int. Cl. A61N 5/10

(52) U.S. Cl. CPC A61N 5/10
USPC 600/1

(57) ABSTRACT

Provided herein are methods for treating neuropsychiatric disorders by delivering radiation to an area in the subgenual anterior cingulate cortex, particularly to the cingulate area 25. Those methods are also beneficial to treat refractory neuropsychiatric disorders.

### 1. CROSS-REFERENCE TO RELATED APPLICATION

This application claims priority and other benefits from U.S. Provisional Patent Application Ser. No. 61/635,033 filed Apr. 18, 2012, entitled “Method of treating a neuropsychiatric disorder using targeted radiation”. Its entire content is specifically incorporated herein by reference.

### 2. TECHNICAL FIELD OF THE INVENTION

The present invention relates to methods of treating refractory neuropsychiatric disorders involving the use of radiation that is targeted to the subgenual anterior cingulate cortex, in particular to the cingulate area 25.

### 3. BACKGROUND

Neuropsychiatric disorders greatly affect the capacity of an individual to socialize, communicate, learn and function independently. Neuropsychiatric disorders that do not respond to standard treatment approaches such as treatment with antidepressants, antipsychotics or anticonvulsants, psychotherapy and, as last resort, electroconvulsive therapy, such as refractory bipolar depression and refractory major depressive disorder, are particularly debilitating to an individual, since they leave that individual without any other option to pursue improvement of his quality of life.

Impairment of neurocognitive functions such as verbal memory, executive and processing functions, social ability and attention is increasingly recognized as a key element in neuropsychiatric disorders including bipolar disorder and schizophrenia (Yatham et al., 2010). An improvement of impaired neurocognitive functions is important in the overall management of neuropsychiatric disorders.

Particular regions of the prefrontal cortex have been found involved in cognitive, emotional and motivational processes. Deep brain stimulation of areas of the subgenual anterior cingulate cortex (subcallosal cingulate gyms), particularly the cingulate area 25, has attracted great interest (Mayberg et al., 2005). Cingulate area 25 is the subcallosal cingulate gyms that includes the Brodmann area 25; it is the part of the cingulum that lies close to the corpus callosum. Imaging studies have detected abnormally high metabolic activity in the cingulate area 25 in subjects suffering from neuropsychiatric disorders, particularly from depression, which was partially reversable by the treatment with various antidepressants.

Once an individual who suffers from a severe neuropsychiatric disorder such as bipolar depression and major depressive disorder fails to recover from a major episode, he is left in a state of extreme depression and lack of executive function, unable to take care of himself in the most basic ways.

The treatment of such severe, often treatment-refractory neuropsychiatric disorders is of immense clinical and humanitarian interest. The present invention addresses this issue.

### 4. SUMMARY OF THE INVENTION

Provided herein are methods for treating subjects who suffer from refractory forms of neuropsychiatric disorders or mood disorders such as depression, bipolar depression, major depressive disorder, schizophrenia, autism and anxiety disorders that do not respond to standard medical treatment.

In one aspect, a therapeutically effective dose of radiation is delivered to an area of the subgenual anterior cingulate cortex of a subject suffering from a neuropsychatric disorder, which might be refractory to treatment, wherein said radiation does not exceed 75 Gray in said area and wherein said radiation is effective to achieve an improvement, as measured using standard protocols for assessing improvement. In one embodiment, a therapeutically effective dose of radiation is delivered to the subject's cingulate area 25.

In another aspect, a therapeutically effective dose of radiation is delivered to an area of the subgenual anterior cingulate cortex of a subject suffering from an impairment of neurocognitive function in the context of a neuropsychatric disorder, which might be refractory to treatment, wherein said radiation does not exceed 75 Gray in said area and wherein said radiation is effective to achieve an improvement of neurocognitive function in the subject, as measured using standard protocols for assessing neurocognitive function. In one embodiment, a therapeutically effective dose of radiation is delivered to the subject's cingulate area 25.

In one embodiment of the present invention, subjects suffering from bipolar depression received a one-time bilateral radiation treatment to the cingulate area 25 (Cg25) using a Cyberknife system (CKS), after magnetic resonance imaging (MRI) and positron emission tomography (PET) results had been obtained for pretreatment planning. Most subjects exhibited a clinical and neurocognitive improvement following treatment that lasted for several months.

The above summary is not intended to include all features and aspects of the present invention nor does it imply that the invention must include all features and aspects discussed in this summary.

### 5. INCORPORATION BY REFERENCE

All publications and patent applications mentioned in this specification are herein incorporated by reference to the same extent as if each individual publication or patent application was specifically and individually indicated to be incorporated by reference.

### 6. BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates for all three subjects the scores from the Hamilton Depression Rating Scale (HDRS-17 item) from screening through the end of study assessment at 12 months.

FIG. 2A illustrates the Clinical Global Impression of Severity (CGI-S) scale outcome scores from screening through the end of study assessment at 12 months.

FIG. 2B illustrates the Clinical Global Impression of Improvement (CGI-I) scale outcome scores from screening through the end of study assessment at 12 months.

FIG. 3 illustrates the YMRS, at the screening visit through end of study assessment at 12 months.

FIG. 4A shows the outcomes for DKEFS neurocognitive testing and CVLT memory test outcomes at baseline, 3 and 12 months for subject 1.

FIG. 4B shows the outcomes for DKEFS neurocognitive testing and CVLT memory test outcomes at baseline, 3 and 12 months for subject 2.

FIG. 4C shows the outcomes for DKEFS neurocognitive testing and CVLT memory test outcomes at baseline, 3 and 12 months for subject 3.

FIG. 5A and FIG. 5B show MADRS scores for Subject 1 and Subject 2 during electroconvulsive treatment. Subject 1 participated in acute and continuation treatments over 6 weeks (FIG. 5B), and Subject 2 over 33 months (FIG. 5A).

FIG. 6A shows the CGI-S clinical rating scores for Subject 1 from clinic assessments in the Bipolar Clinic, spanning the time period from Jun. 11, 2003 to 2012. There were a total of 116 visit assessments over the 6 years and 9 months prior to the CKS procedure, 21 visits during the 12-month study follow-up period and 66 visits in the 21 months post-CKS.

FIG. 6B shows the CGI-S clinical rating scores for Subject 2 from clinic assessments in the Bipolar Clinic, spanning the time period from Apr. 8, 2002 and continuing to 2012. There were a total of 230 visit assessments for the 8 years and 2 months leading up to the CKS procedure, 30 visits during the study period, and 25 visits in the 12 months post-CKS. A dashed trend line in red shows the overall course of illness during this time period.

### 7. DETAILED DESCRIPTION

In one aspect, the present invention provides a method of treating subjects who suffer from a neuropsychiatric disorder with low-dose radiation not exceeding 75 Gray that is targeted to an area of the subgenual anterior cingulate cortex, particularly to the cingulate area 25.

In another aspect, the present invention provides a method of treating subjects who suffer from a refractory neuropsychiatric disorder with low-dose radiation not exceeding 75 Gray that is targeted to an area of the subgenual anterior cingulate cortex, particularly to the cingulate area 25. In one embodiment of the present invention, subjects suffering from refractory bipolar depression received a one-time bilateral radiation treatment to the cingulate area 25 (Cg25) using a Cyberknife system (CKS), after magnetic resonance imaging (MRI) and positron emission tomography (PET) results had been obtained for pretreatment planning. Most subjects exhibited a clinical and neurocognitive improvement following treatment that lasted for several months.

This approach is considered particularly useful for subjects who suffer from refractory forms of neuropsychiatric disorders that do not respond to standard treatment.

Before describing detailed embodiments of the invention, it will be useful to set forth definitions that are utilized in describing the present invention.

### 7.1. DEFINITIONS

The practice of the present invention may employ conventional techniques of neurochemistry, neuroscience, immunohistochemistry and molecular biology, which are within the capabilities of a person of ordinary skill in the art. Such techniques are fully explained in the literature. For definitions, terms of art and standard methods known in the art, see, for example, Sambrook and Russell “Molecular Cloning: A Laboratory Manual”, Cold Spring Harbor Laboratory Press (2001); ‘Current Protocols in Molecular Biology’; ‘Current Protocols in Immunology’; Stanley ER “Colony stimulating factor-1”, The cytokine handbook, 1994, Academic Press. Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “subject”, as used herein, refers to a mammal, including mouse, rabbit, dog, cat, guinea pig, goat, cow, horse, pig, sheep, monkey, primate, ape, and, preferably, a human.

Neuropsychiatric disorders or mood disorders, as used herein, include depression, bipolar depression, major depressive disorder, schizophrenia, autism, anxiety disorders, and the like.

By impaired neurocognitive function is meant an impairment with a pattern of persistently low ability to memorize facts, impairment in learning ability, impairment in emotional processing.

Targeted radiation, as used herein, is carried out by directing a high energy beam of radiation on a target, a concept that is known as stereotactic radiosurgery. Alternatively, a series of radiation beams can be converged from various angles on the same target to achieve linear accelerator-based stereotactic radiosurgery (Hoffelt, 2006).

Stereotactic Radiosurgery.

In one embodiment, a Cyberknife System™ (CKS) was used to deliver targeted radiation to the cingulated area 25. Other methods to deliver targeted radiation include stereotactic radiosurgery units such as the Gamma Knife™. In comparison to the Gamma Knife™, the Cyberknife System™ uses real-time X-ray images to guide treatment (Hoffelt, 2006).

The terms “neurocognition” and “neurocognitive function”, as used herein, refers to a subject's ability to store and retrieve memories, pay attention, communicate and function independently.

The term “treatment refractory”, as used herein, means unresponsive or resistant to (i) treatment with one or more therapeutic agents that are characterized as having an anti-depressive, anticonvulsant or anti-psychotic effect, (ii) psychological therapy and/or (iii) electroconvulsive therapy (ECT). Treatment refractory neuropsychiatric disorders are severely disabling disorders that no longer have an effective treatment option available. Neuropsychiatric disorders are treatment refractory, once treatment with one or more anti-depressive or antipsychotic agents, psychological therapy and/or electroconvulsive therapy has failed to show an improving effect.

A therapeutically effective dose of targeted radiation is defined as an amount of radiation that shows an improvement of clinical parameters and/or neurocognition in the subject that received the radiation. Such targeted radiation may be administered in a single dose, in a single daily dose, or the daily dose may be administered in divided doses of two, three, or more times per day. Furthermore, the total dose may be administered in divided doses of two, three, or more times over a period of time ranging from several days, weeks, months to years.

The term “therapeutic effect”, as used herein, refers to a consequence of treatment that might intend either to bring remedy to an injury that already occurred or to prevent an injury before it occurs. A therapeutic effect may include, directly or indirectly, the improvement of a subject's neurocognitive function, as evidenced by an improvement of the subjects' ability to store and retrieve memories, pay attention, communicate, socially interact and function independently. A therapeutic effect may also include, directly or indirectly, the improvement of symptoms of a neuropsychiatric disorder, as evidenced on improvements on standard tests.

### 7.2. Neuropsychiatric Disorders (Mood Disorders)

Bipolar Disorder (Bipolar Depression, BD).

Bipolar disorder is a chronic and recurrent neuropsychiatric disorder that causes extraordinary mood shifts in a subject and that is characterized by impairments in neurocognitive functions (neurocognition), particularly in the areas of verbal memory, executive functions, social ability and attention (Sole et al., 2012).

Bipolar disorder has an illness course broadly characterized by recurrent and chronic major depressive episodes (MDE) as the illness progresses. Patients with Bipolar 1 disorder (BP1) have euphoric manias early in the course, while over time these euphoric states are replaced by dysphoric or mixed manic states, rapid cycling, and severe, chronic MDEs that are difficult to treat. Bipolar 2 (BP2) has a similar illness course, but with hypomanic episodes which are not as severe or impairing as full mania, though the depressive phase of the illness is just as severe, recurrent and chronic as for BP1.

When in the depressive phase, unipolar and bipolar depression are symptomatically indistinguishable. However, those with BP disorder are more likely to have a history of a psychotic MDE, a family history of bipolar disorder, and an early onset of illness. Bipolar depression is often under- or misdiagnosed among the population of those thought to have treatment resistant unipolar depression. When all three of the above criteria were met, a careful assessment using the Structured Clinical Interview for Diagnosis (SCID) revealed that 85% of those previously diagnosed with major depression were found to have BPD. Resistant/refractory BPD is highly prevalent among treatment resistant depressed patients.

Approximately two thirds of patients with bipolar depression fail to recover from a major depressive episode, even on complex medication regimens. Electroconvulsive therapy (ECT), where a small amount of electricity is applied to certain brain areas to produce a mild generalized seizure or convulsion, is the treatment of last resort after all other aggressive therapies have failed and the subject requires immediate intervention due to a high propensity to suicide and self-injury. While highly effective in the acute treatment of depression, between 15 and 50% do not respond and relapse is common. Subjects with treatment resistant bipolar disorder have a notably higher risk of morbidity and mortality.

Major Depressive Disorder (MDD).

Major depressive disorder, also known as clinical depression, major depression, unipolar depression or unipolar disorder, is, besides bipolar disorder, the most frequently occurring form of a mood disorder. Associated with a high level of morbidity and mortality, major depressive disorder is characterized by low self-esteem, lack of interest in social contacts and in otherwise enjoyable activities and a state of low mood (Lohoff, 2010).

Schizophrenia.

Schizophrenia is a chronic, severe and disabling neuropsychiatric disorder that manifests itself with halluzinations, delusions and thought as well as movement disorders. Subjects who are affected by schizophrenia usually exhibit abnormal emotions and behaviors and poor executive functioning abilities (Haskins et al., 1995).

Anxiety Disorders.

Anxiety disorders are chronic neuropsychiatric disorders that are characterized by excessive worrying and apprehension about future events and situations which may affect a subject's physiological, emotional/behavioral and psychological health. ‘Anxiety attacks’ can be triggered in times of high stress. Anxiety disorders are classified in various categories, including, but not limited to, generalized anxiety disorder, phobic disorder, panic disorder, agoraphobia, social anxiety disorder, obsessive-compulsive disorder, post-traumatic stress disorder, separation anxiety and situational anxiety.

Autism.

Autism is a behavioral disorder which develops in early age and which is defined on the basis of impairments in social interaction and communication and characterized by repetitive and stereotypic behavior and use of ideosyncratic language. Depending on the severity of autism, children who are of the low-functioning type of autism are generally mentally retarded to a certain degree, as indicated by IQ testing.

Most children with autism fail to develop reciprocal communication and cannot interpret social cues. While these children often possess good expressive language, they have large deficits in receptive language. Like Autism, Rett syndrome belongs to the umbrella category of pervasive developmental disorders and exhibits remarkable phenotypic overlap with autism.

### 7.3. Subgenual Anterior Cingulate Cortex

Particular regions of the prefrontal cortex have been found involved in cognitive, emotional and motivational processes. The dorsolateral frontal cortex projects to the dorsolateral head of the caudate nucleus and has been linked to executive functions such as verbal fluency, planning, working memory, organizational skills, reasoning, problem-solving and abstract thinking (Alvarez & Emory, 2006).

The subgenual anterior cingulate cortex (ACC), which is ventral to the corpus callosum, belongs to the brain's limbic system and is the frontal part of the cingulate cortex. The anterior cingulate cortex is a part of a neuroelectrical circuit that is involved in cognitive and emotional processing (Bush et al., 2000; Drevets et al., 2008). The ACC is involved in the control and regulation of autonomic functions such as blood pressure and heart rate as well as in the regulation of cognitive functions and emotional experiences. Such cognitive functions include good decision making, motivated attitude, and impuls control. The involvement of the subgenual anterior cingulate cortex (subcallosal cingulate gyms) in approaches to treat depression and its potential relevance as a target for deep brain stimulation has attracted great interest (Mayberg et al., 2005).

Cingulate 25.

Cingulate 25 (Cg 25), a part of the subgenual anterior cingulate cortex, is considered an important nexus in a web of connected prefrontal, subcortical, temporal, and limbic structures. Cingulate 25 dysregulation has been implicated to be a key feature of the pathophysiology of neuropsychiatric disorders. Morphologically, there is significant atrophy of Cg25, with glial loss being the most prominent finding. When a subject is in a depressed state, Cg25 has been found to be in a state of pathological hypermetabolic activity, which is normalized in those subjects who respond to antidepressant treatment.

With bipolar depression it has, furthermore, been found that a progression of illness is associated with plastic change and apparent atrophy in key neural circuitry that impairs neurocognitive function such as prefrontal cortical processing efficiency. Impairment of many functions sub-served by these prefrontal cortical regions that are dysregulated in bipolar disorder contribute to the persistent disability and neurocognitive impairment observed even when patients are euthymic.

### 7.4. Targeted Radiation: Radiosurgical Neuromodulation

Low dose radiation treatment has been used extensively and successfully for the treatment of trigeminal neuralgia, with a low incidence of facial sensory loss as a result of treatment. Clinical outcomes for treatment using both the gamma-knife system (GKS) and CyberKnife System (CKS) have been published. Though limited, the data to date in animals and in humans show that radiation targeting the proximal nerve root with doses up to 80Gy does not cause neuronal cell death in the retrogassian ganglion. It does cause extensive damage to myelin along the section of the proximal axon where the radiation is focused. In animal studies a dose of 100 Gy will cause necrosis, with 90 Gy being intermediate in terms of damage to neuronal cell bodies, and destruction of white matter along the nerve length.

Treatment refractory trigeminal neuralgia has been responsive at non-ablative doses averaging about 60 Gy, with a maximum dose of 75 Gy. Patients have reduced facial pain, without any evidence of radionecrosis in the retrogassian trigeminal nerve.

Low-Dose Radiation (Around 60 Gy) Targeted to Areas of the Subgenual Anterior Cingulate Cortex, Particularly to Cingulate 25 (Cg-25).

Low dose radiation, not exceeding 75 Gray (Gy) per radiation cycle, can modulate precisely targeted small volume bran regions and can change neural function without necrosis. The Gray measures the absorbed energy of radiation.

As described in Example 1, the inventors of the presently described invention hypothesized that precisely targeted low dose radiation could reduce local metabolic activity in Cg25, and reduce the severity of depression without causing worsening of symptoms or disability. This approach using low dose radiation to modulate neuronal function and to alter circuit behavior without causing cell death is referred to as Radiosurgical Neuromodulation (RSN).

The CyberKnife System (CKS) and gamma knife are well-known devices to apply stereotactic radiosurgery. Both systems have been used to treat trigeminal neuralgia pain with precise delivery of low dose radiation to the proximal axon of the trigemineal nerve. Some have suggested that the absence of necrosis in the ganglion is evidence that such an approach can selectively induce a functional ablation of pain fibers transmission, without cell death.

CyberKnife System.

The CyberKnife is a stereotactic radiosurgery instrument that enables large doses of radiation to be cross fired through and concentrated in a specific small brain target with very high (sub millimeter) accuracy. The Cyberknife targets the delivery of radiation which is emitted by a 6 MV Linear accelerator by using x-ray image to image correlation; live projection x-rays are compared with a patient's prior CT scan. Alternative radiosurgical instruments (The GammaKnife, Novalis etc.) also enable the accurate administration of high dose radiosurgery through the use of steroetactic frames attached to the patient's skull. These devices are capable of treating refractory neuropsychiatric disorders with radiation.

Deep brain stimulation in cingulate 25 (Cg25) using implanted electrodes is currently being investigated for efficacy in treatment refractory unipolar and bipolar depression (Mayberg et al., 2005). This target has been identified as a key structure within multiple neural circuits whose dysfunction is implicated in the neurobiology of depression. Deep brain stimulation is thought to reduce and normalize local metabolism in Cg25, resulting in normalization of circuit behavior, and an improvement in depressive symptoms.

### 7.5. Assessing Improvement Using Standard Protocols/Tests

An improvement of symptoms of a neuropsychiatric disorder or lack of improvement was evaluated using standard tests such as the 24-item Hamilton Depression Rating Scale (HDRS) for depression, while manic symptoms were analyzed with the Young Mania Rating Scale (YMRS), and the Clinical Global Impression of Severity of Illness (CGI-S) as well as the Clinical Global Impression of Improvement of Illness (CGI-I). Memory function was assessed with the California Verbal Learning Task (CVLT), while prefrontal function was evaluated using a set of Delis-Kaplan Executive Function System (D-KEFS) tests including the D-KEFS Sorting task, D-KEFS Trails task, D-KEFS color-word interference task, and the D-KEFS verbal fluency task.

Hamilton Depression Rating Scale (HDRS).

The Hamilton Depression Rating Scale, also known, as Ham-D, is a widely used clinician-administered and clinician-rated questionnaire to assess the severity of a subject's depressive symptoms and any changes thereof (Hamilton, 1960). The HDRS is designed for adults and contains questions relating to mood, feelings of guilt, suicidal thoughts, insomnia, agitation or retardation, anxiety, weight loss and somatic symptomes. In its original form, the HDRS consisted of 17 question items, but various versions with up to 29 question items exist as well.

Young Mania Rating Scale (YMRS).

The Young Mania Rating Scale is an 11-item, clinician-administered and clinician-rated questionnaire to assess the severity of mania and any changes thereof (Young et al., 1978). The YMRS was originally assigned for adults, but has also been adapted for pediatric subjects; its questions relate to mood, motor activity, sleep requirements, disruptive-aggressive behavior, irritability, appearance, rate and amount of speech and insight.

The Clinical Global Impressions Scale of Improvement of Ilness (CGI-I) and the Clinical Global Impressions Scale of Severity of Illness (CGI-S).

The CGI provides clinicians a tool for a brief assessment of a subject's global functioning prior to and after initiating a study medication. This information includes a subject's history, psychosocial circumstances, symptoms, behavior, and the impact of the symptoms on the patient's ability to function. It is applicable to many neuropsychiatric disorders, including depression, schizophrenia and anxiety (Busner & Targum, 2007).

The California Verbal Learning Test (CVLT) is a neuropsychological test that is used to assess a subject's verbal learning and verbal memory abilities. In addition to recall and recognition scores, it measures encoding strategies, learning rates, error types, and other process data (Delis et al., 2000).

The Delis-Kaplan Executive Function System (D-KEFS) tests assess executive functions, i.e. higher-level cognitive functions, in children, adolescents, and adults of all ages. Executive functions encompass neurocognitive processes that regulate, control and manage a variety of cognitive processes such as executive functions including flexibility of thinking, inhibition, problem solving, planning, impulse control, concept formation, abstract thinking, and creativity (Homack et al., 2007). The California Verbal Learning Test and the Delis-Kaplan Executive Function System tests are also suitable for evaluating a subject for an improvement in neurocognitive function.

An improvement of neurocognitive function can, furthermore, be evaluated using any suitable standard protocol for assessing neurocognitive function, including, but not limited to, the Wechsler Adult Intelligence Scale and the Mini-Mental State Examination (MMSE).

Wechsler Intelligence Scales.

The Wechsler Intelligence Scales (Wechsler, 1981). were designed to measure intelligence in adolescents and adults (Wechsler Adult Intelligence Scale), but also in children (Wechsler Intelligence Scale for Children). These tests analyze a subject's general knowledge, language, reasoning, memory skills, spatial, sequencing, and problem-solving skills.

Mini-Mental State Examination.

The mini-mental state examination is a short questionnaire to assess cognitive functions, estimate the severity of cognitive impairment and to document any cognitive changes (Folstein et al., 1975).

By improvement is meant at least an amelioration of the symptoms, where amelioration is used to refer to at least a reduction in the magnitude of a parameter, e.g. a symptom, associated with the pathological condition being treated, such as impairment in memory or learning ability or other neurocognitive function. Treatment or improvement also includes situations where the pathological condition, or at least symptoms associated with, are completely abrogated, e.g., prevented from occurring, or terminated such as that the subject no longer suffers from the pathological condition, or at least the symptoms that characterize the pathological condition.

As mentioned above, in these applications a therapeutically effective amount of low-dose radiation is administered to an area of the subgenual anterior cingulate cortex, particularly the cingulate area 25 (Cg-25) area of the subject. By “therapeutically effective amount” is meant a radiation dose sufficient to produce a desired result, where the desired result is generally an amelioration or alleviation, if not complete cessation, of one or more symptoms of the neuropsychiatric disorder being treated, particularly the neurocognitive impairment symptoms, symptoms of extreme depression, dispair and hopelessness. In such methods, a low-dose radiation was administered to an area of the subgenual anterior cingulate cortex of a subject, particularly to the cingulate area 25 (Cg-25) area, typically according to a dosing schedule, e.g. daily, weekly, monthly and so forth, that is sufficient to ameliorate the neuropsychiatric disorder or at least symptoms of the neuropsychiatric disorder and to improve neurocognitive function, where impaired.

In addition to the above methods of treatment, the subject methods also find use in the prophylactic or preventative treatment of neuropsychiatric disorders. In such methods, a low-dose radiation is administered to an area of the subgenual anterior cingulate cortex of a subject, particularly to the cingulate area 25 (Cg-25) area, typically according to a dosing schedule, e.g. daily, weekly, monthly and so forth, that is sufficient to prevent the occurrence of at least symptoms of the neuropsychiatric disorder.

### 7.6 Methods

In the broadest sense, methods are provided for treating a neuropsychiatric disorder or at least ameliorating symptoms thereof and, in case of impairment, for improving neurocognitive function in a subject. The subject is generally a mammal, e.g. mouse, rat, monkey, etc. and in preferred embodiments a human. The low-dose radiation to the subject's subgenual anterior cingulate cortex, particularly to the cingulate area 25, is administered either just once or at regular intervals, usually at least monthly, weekly, daily, or every few days.

Treatment Planning.

Prior to the administration of radiation, a treatment planning step can be carried out using images, for example from magnetic resonance imaging (MRI) and positron-emission-tomography-computed tomography (PET-CT) scans, to find the volume of the target location and appropriate dose within the Cingulate area 25 or other area within the subgenual anterior cingulate cortex that should be targeted with the low-dose radiation.

Schedule of Administration of Low-Dose Radiation.

Administration of the low-dose radiation treatment is maintained for a period of time sufficient to effect a therapeutically desired change, as evidenced by an amelioration of symptoms of the neuropsychiatric disorder and/or by an improvement in neurocognitive function. Such treatment may involve administering low-dose radiation (up to 75 Gray) to the subject's cingulate area 25 for at least once, or for about a few days or about a week; at least about two weeks; at least about 3 weeks; at least about one month; at least about two months; at least about four to six months; or longer, for example at least about one or more years. For extended treatment; e.g. treatment of one or more years, a schedule may involve intermittent periods, such as one week on and one week off; two weeks on and two weeks off; one week in a month, etc.

Low-Dose Radiation.

Low doses of radiation are administered in each application unit, whereby the minimum dose is 1, 2, 3, 4, 5, 6, 7, 8, 9 or 10 Gray and the maximum, not to be exceeded, dose is 75 Gray.

Subjects who can benefit from the present invention may be of any age and include adults and children, including young adults.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### 8. EXAMPLES

The following examples are put forth to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Example 1

### Treatment of Refractory Bipolar Depression Using Low Dose Radiation Targeting Cingulate 25

An Investigational Device Exemption (IDE: G090015) was obtained from the Food and Drug Administration for a safety and feasibility trial to test whether radiosurgical neuromodulation (RSN) targeting cingulate area 25 (Cg 25) with low-dose radiation, as defined earlier, would decrease local metabolic activity in Cg25, and reduce depressive symptoms in patients with refractory bipolar depression, as assessed by standard protocols. The radiosurgical neuromodulation procedure was carried out using the CyberKnife System (CKS).

The CyberKnife System.

The CyberKnife is a stereotactic radiosurgery instrument that enables large doses of radiation to be cross fired through and concentrated in a specific small brain target with very high (sub millimeter) accuracy. The Cyberknife targets the delivery of radiation which is emitted by a 6 MV Linear accelerator by using x-ray image to image correlation; live projection x-rays are compared with a patient's prior CT scan

While any radiosurgical device can be used, the research team had extensive experience with the Accuray CyberKnife System, K011024, and it was, therefore, used for planning and delivery of the 6MV X-ray treatment for this study.

In the following, outcomes are reported for three subjects who underwent the CKS procedure and were assessed over 1 year using sequential mood scales, neurocognitive testing, and extensive structural MRI as well as functional imaging.

Methods.

Each subject was carefully screened and assessed, then a detailed discussion of the study, the potential risks and benefits ensued, followed by specific questions by the subject, until they fully understood the study, the procedures, the potential risks and possible benefits of the CKS procedure. The subjects were assessed by a psychiatrist (David Speigle MD) to verify the competence of the subject to understand the risks and benefits of participation in the study, and to uncover special circumstances that might have created a context of despair or desperation that could unduly influence the decision by the subject to participate in the trial. The inclusion criteria for the study are found in Table 1 and the schedule of events and assessments in Table 2.

TABLE 1
Inclusion and Exclusion Criteria

Inclusion criteria
Men and women 22-65 years of age
Primary diagnosis of bipolar depression per DSM IV-TR criteria
Duration of the major depressive episode is more than 1 year
HDRS-24 item greater than or equal to 20
Negative urine pregnancy in female subjects
No comorbid post traumatic stress disorder, the inclusion of subjects with other psychiatric
comorbidity will be determined by the investigator prior to enrollment
No personality disorder that in the opinion of the investigator may compromise the subjects
ability to participate and be compliant with the elements and procedures of the study
No substance abuse of dependence in the last 6 months
A negative urine drug screen
On a stable dose of their current medication regimen for four weeks
Treatment resistance: A history of failure to show clinical improvement after at least four
different medication trials of adequate duration and dose and one course of ECT. A course
of ECT is not mandatory for those who have declined the procedure due to stigma, patient
preference, or intolerance. One course of ECT is defined as receiving six acute treatments.
(Note: the Antidepressant Treatment History Form (ATHF) does not apply to bipolar
depression where mood stabilizers are often the treatments of choice).
No available reasonable treatment options at the time of enrollment
Competent to understand the risks and potential benefits of the study
Able to provide written informed consent for the full screening phase, as well as the
treatment period of the protocol, including the baseline MRI, CT and PET imaging
Signed consent form for participation in the study
Exclusion criteria
Rapid cycling bipolar illness
History of schizophrenia, schizoaffective disorder, or psychosis
Severe suicidal thoughts that may put the subject at risk of either an attempted suicide or
completed suicide for the duration of the trial, as determined by the investigator at the time of
enrollment
Current substance abuse or in the process of withdrawal from mind-altering substances
including alcohol, stimulants, or sedatives
Undisclosed or undiagnosed unstable medical or neurologic illness including stroke,
significant brain malformation, or brain mass
Previous whole-brain radiation
Brain-implanted devices such as deep brain stimulation leads or aneurysm clips
A history of seizure disorder
History of brain injury
Current treatment with either metronidazole or cisplatinum and an inability to discontinue
prior to RSN
Pregnancy or breast feeding

TABLE 2
Treatment Protocol

| | | Base- | Week | | | | | Month | | |
|---|---|---|---|---|---|---|---|---|---|---|
| | Screen | line | 1 | 2 | 4 | 8 | 12 | 6 | 9 | 12 |
| Informed | X | | | | | | | | | |
| Consent | | | | | | | | | | |
| Medical | X | | | | | | | | | |
| History/ | | | | | | | | | | |
| Psychiatric | | | | | | | | | | |
| History | | | | | | | | | | |
| Current | X | X | X | X | X | X | X | X | X | X |
| medications | | | | | | | | | | |
| Psychiatric | X | X | X | X | X | X | X | X | X | X |
| evaluation | | | | | | | | | | |
| Urine | X | | X | | | | | | | |
| pregnancy | | | | | | | | | | |
| test | | | | | | | | | | |
| Clinical | X | X | X | X | X | X | X | X | X | X |
| Scales: | | | | | | | | | | |
| HDRS, | | | | | | | | | | |
| YMRS, | | | | | | | | | | |
| MADRS; | | | | | | | | | | |
| CGI-S (I) | | | | | | | | | | |
| Self-Report | X | X | X | X | X | X | X | X | X | X |
| Forms | | | | | | | | | | |
| Neuropsych: | X | | | | | | X | | | X |
| CVLT, | | | | | | | | | | |
| DKEFS | | | | | | | | | | |
| MRI | X | | X | | | | X | X | | X |
| PET/CT | X | | | | | | X | | | |
| Adverse | | X | X | X | X | X | X | X | X | X |
| Events | | | | | | | | | | |
| logged | | | | | | | | | | |
| RSN | | X | | | | | | | | |
| Treatment | | | | | | | | | | |

All subjects were interviewed using the MINI SCID to confirm the DSM-IV diagnosis of a major depressive episode. Clinical rating scales used were: the 24 and 21 item Hamilton Depression Rating Scale (HDRS), the Clinical Global Impression of Severity (CGI-S) and improvement (CGI-I), and the Young Mania Rating Scale (YMRS). These clinical assessments were done at weeks 1, 2, 4, 8, and 12 weeks post RSN and at the 6, 9, 12 months post-RSN follow up. Prior to receiving the RSN procedure, patients were administered neuropsychiatric testing using the D-KEFS and CVLT. These tests were also repeated at the 3-month and 12-month follow-up visits. Adverse events were reviewed and logged with each visit.

Subjects 1 and 2 had been seen in the Bipolar Clinic and had CGI-S and GAF ratings at each visit as part of the STEP-BD study. These clinic assessments were done by a single clinician for each subject, and were continued during ECT treatment, and after they enrolled in the CKS study, with additional follow up assessments for greater than a year after the study was completed. This provided unique longitudinal data over 10 years for subject 1 (201 assessments), and 11 years for subject 2 (274 assessments).

Following magnetic resonance imaging (MRI) and positron-emission-tomography-computed tomography (PET-CT), the target location within Cingulate area 25 was determined using a fusion of the images from the MRI and PET/CT scans using the standard CyberKnife treatment-planning interface. The target region was approximately 5 mm in diameter and was less than one cubic centimeter in volume. The target was constructed bilaterally in the right and left hemispheres. The software computed an optimal set of beam delivery trajectories in which a dose of 60 Gray (Gy) was prescribed to the target volume, with a maximum dose at any point of 75 Gy.

Administration of low-dose (60-75 Gy) radiation using the CyberKnife System (CKS). Radiation was administered to the left and then right Cg25 target over consecutive days. During the procedure the subjects were placed in a supine position on a CyberKnife treatment table with their head immobilized using a customized headrest and facemask. The subjects were then registered with the stereotactic coordinates of the CyberKnife System, using the standard X-ray camera/DRR (Digitally Reconstructed Radiograph from the CT volume) matching system. Once proper registration was confirmed, radiation delivery began in accordance with the treatment plan. Sixty Gy (up to a maximum of 75 Gy) were delivered to the Cg25 target through a 5 mm collimator. Following the treatment, the subjects were given 4 mg of dexamethasone to minimize the likelihood of acute nausea associated with the treatment (standard practice at Stanford). The subjects were then discharged with follow-up instructions. A brief physical exam was conducted at the first follow-up appointment one week after the CKS procedure by a neurosurgeon.

Imaging.

Ninety days post-RSN treatment, the subjects were re-evaluated using clinical scales for depression, but also by another set of cognitive and memory tests. Follow-up MRI, CT and PET imaging studies were carried out as well to carefully examine whether changes in white or gray matter were detectable compared to the previous scans. The MRI included T1 and T2 images with and without contrast SPGR sequence to detect radiation-induced structural changes. The data and progress of subjects were reviewed every three months including clinical status, test results, imaging data, and assess any adverse events.

Results for Subject 1 (Male).

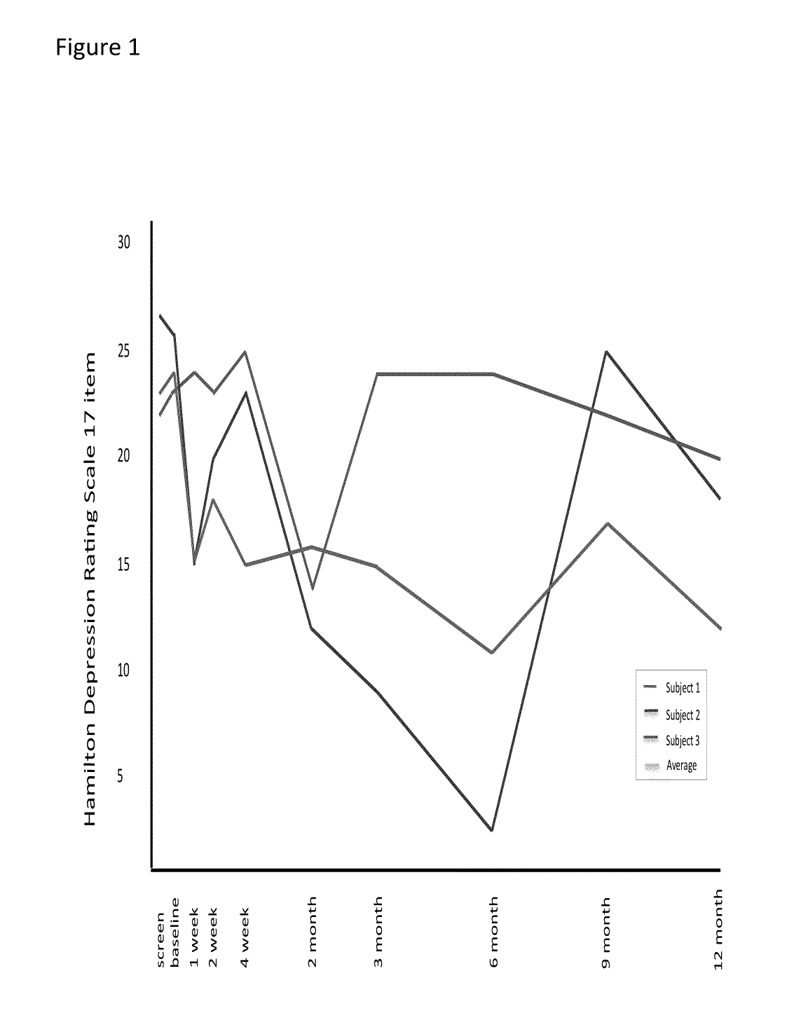

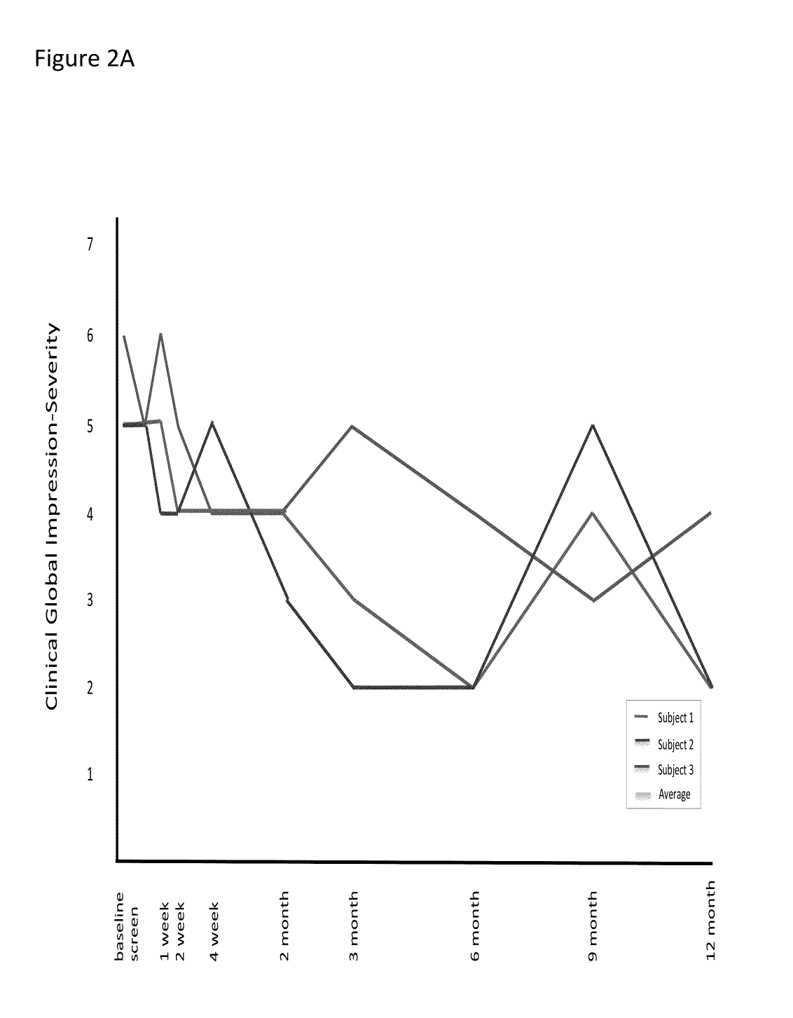

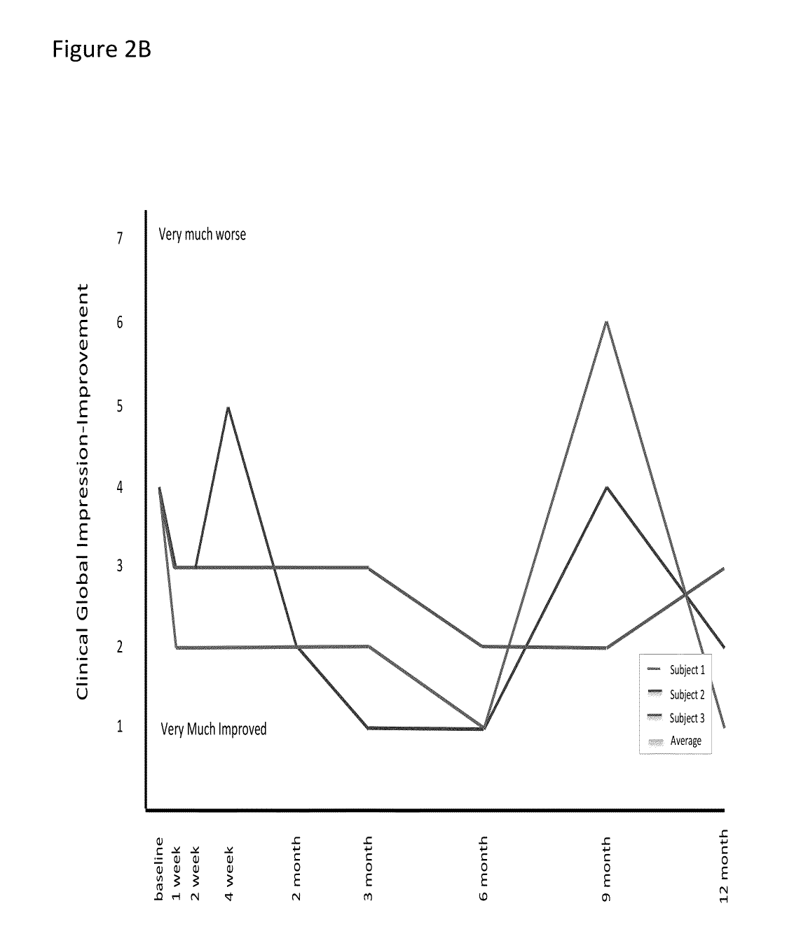

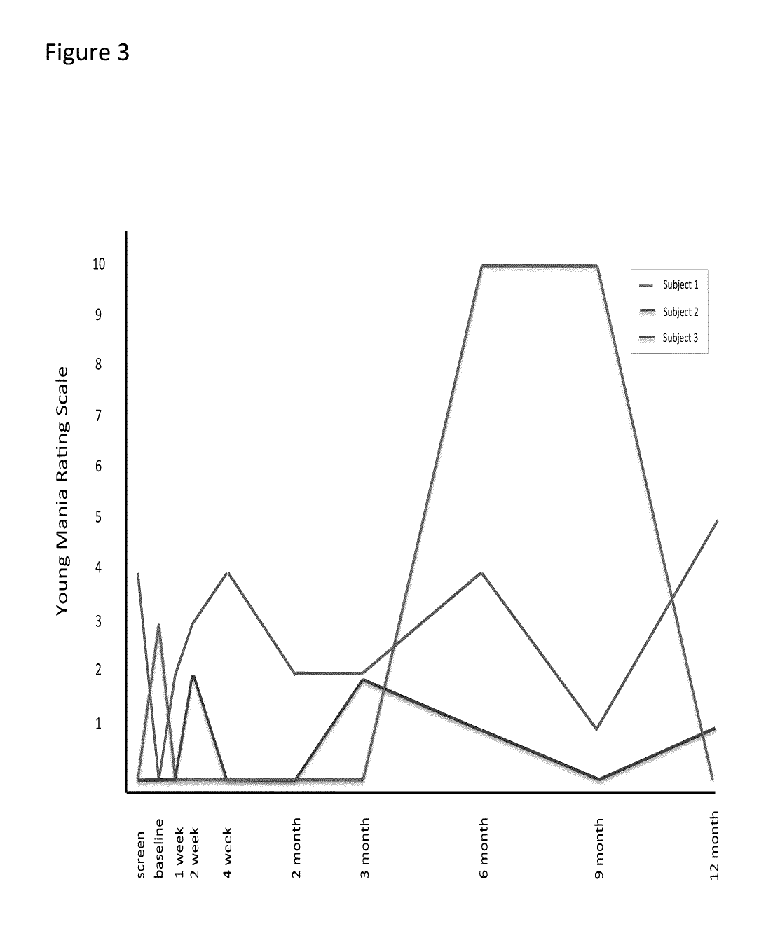

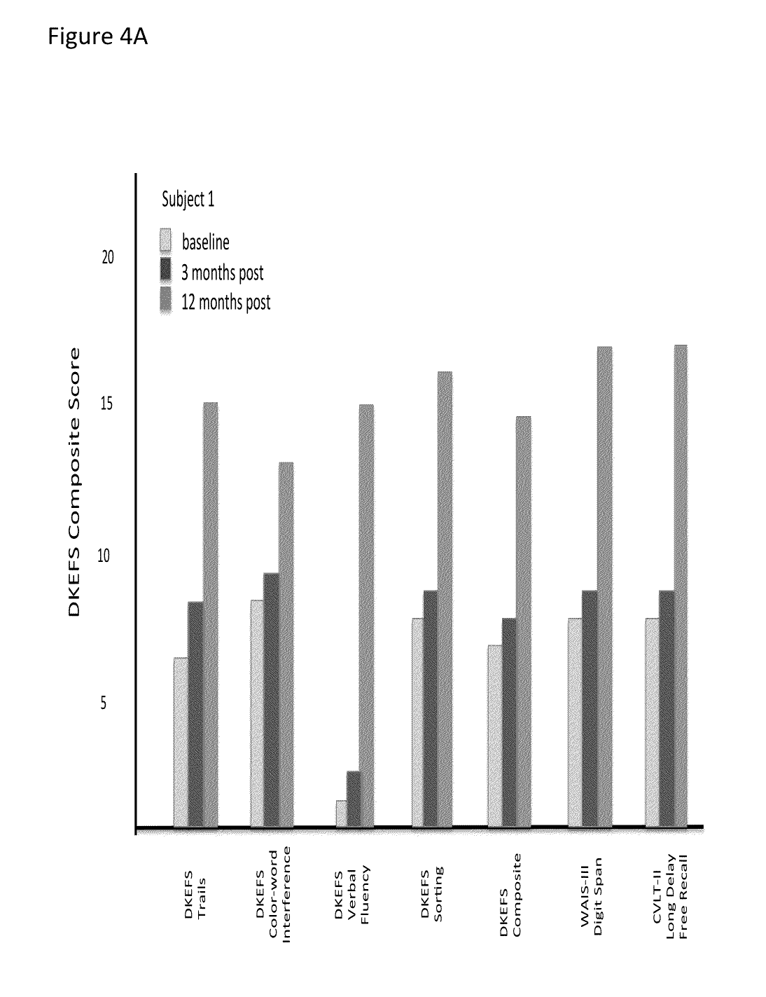

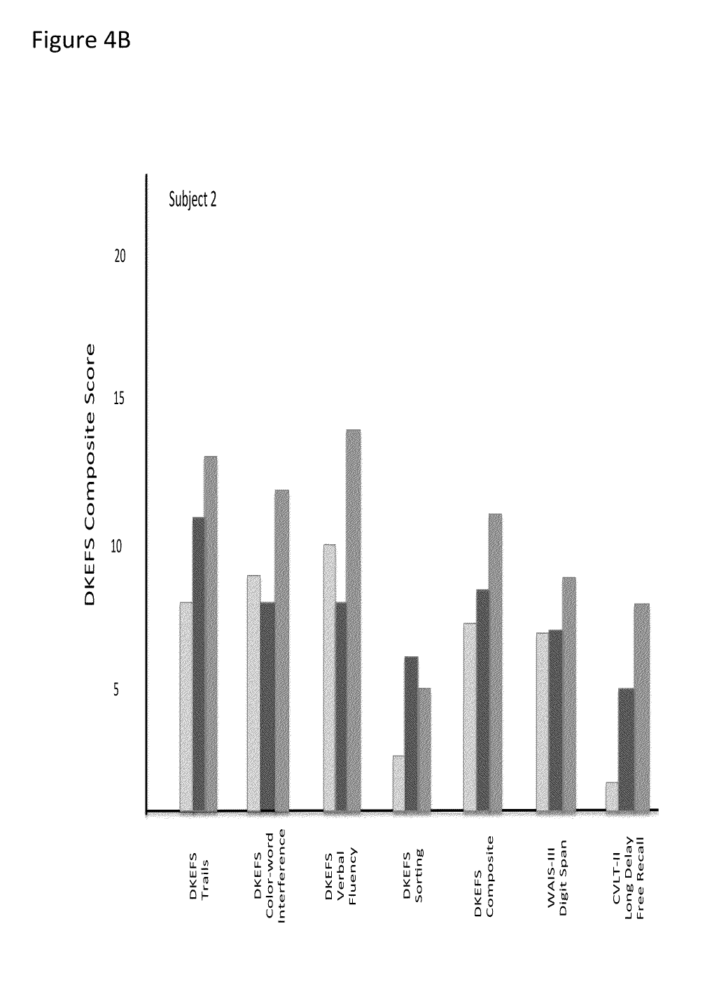

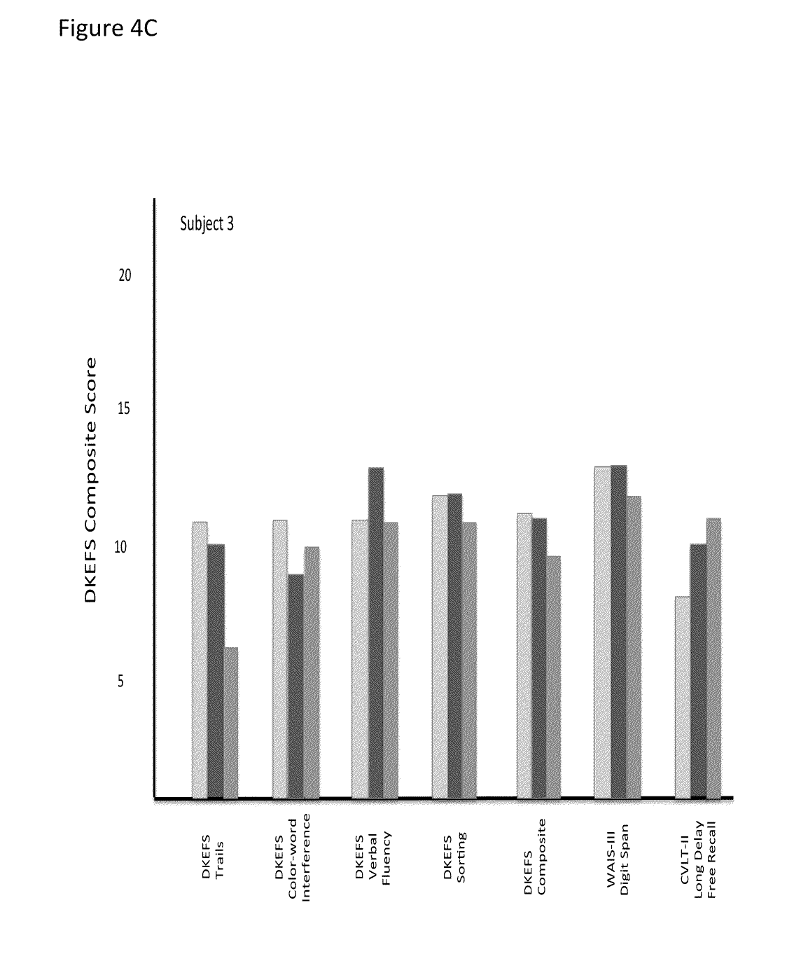

There was substantial variability over the 12 months post-procedure among the subjects. All subjects showed a similar pattern of improvement up to the 3-month time point, while only subjects 1 and 2 continued to follow this trend to the 6-month assessment. Subject 1 met response criteria (>=50% reduction) on the Hamilton Depression Rating Scale (HDRS) 17 at 6 and 12 months, and showed a decrease of 54% and 50% respectively (FIG. 1). He was a responder on the HDRS-24 at 3 months and on both the HDRS-21 and 24-item scales at 6 and 12 months (not shown). As illustrated in FIG. 2, Subject 1 had a drop on the Clinical Global Impression of Severity (CGI-S) from 5 at baseline (markedly depressed) to 2 (borderline mentally ill) at the 6- and 12 month assessments, and the Clinical Global Impression of Improvement (CGI-I) was 1 (very much improved). The Young Mania Rating Scale (YMRS), shown in FIG. 3, increased at 6 and 9 months corresponding to the switch into hypomania and then mania (at 9 months) after the subject self-discontinued a mood stabilizer.

Results for Subject 2 (female). Subject 2 showed a sustained improvement in depressive symptoms from the 2 to 6 month time points. During this period she met response criteria at months 2 and 3, and achieved remission at 6 months (defined as a HDRS 17<=7) with an HDRS-17 score of 2 (FIG. 1). At 3, 6 and 12 months the CGI-S dropped from 5 (severely depressed) to 2, and the CGI-I was 1 at 6 and 12 months (FIG. 2). As described in detail below, an unexpected stressful event (suicide of her close cousin, who had grown up like a sister to her) precipitated a decline at the 9-month assessment, but she recovered much of her earlier improvement by the end of the study. Over the entire 12-month period subject 2 showed a decrease in the HDRS-17 of 31%.

Results for Subject 3 (Male).

Subject 3 showed a pattern of decreasing severity of symptoms to 3 months but then returned to baseline (FIG. 1). The improvement seen over the entire 12 months following the CKS procedure on the CGI-S and I scales (FIG. 2) was reflected by improvements in his quality of life, in his ability to work, his ability to hold a conversation and to problem solve, and his participation in social events that he reported as very enjoyable and distinct from his pre-CKS baseline. Despite reported functional improvements and apparent improvements in his quality of life, the depressive symptoms assessed on the Hamilton Depression Rating Scale (HDRS) did not follow this pattern of change.

Improvement in Neurocognitive Function.

Neurocognitive outcomes are shown in FIG. 3 for all 3 subjects. The Delis-Kaplan Executive Function System (DKEFS) scores were composite scores for each type of assessment battery: trails, color-word interference, verbal fluency, sorting, and an overall composite score. The Wechsler Adult Intelligence Scale (WAIS)-III digit span and California Verbal Learning Test (CVLT)-II long delay free recall outcomes followed the DKEFS scores. Serial testing at baseline, 3 and 12 months showed that subject 1 appeared to have a marked improvement from baseline to the end of 12-months. Subject 2 also showed some improvement in performance over the 12-month follow up period. Subject 3 appeared to perform less well on the DKEFS trails at the end of the study, but otherwise he showed no change across the remainder of the battery.

Interpreting the outcomes for subject 1 and 2 over the entire 12-month follow up period was complicated in each case by a circumstance that directly and negatively affected a consistent pattern of improvement seen over the first 6 months. Just after the 6-month assessment, subject 1 discontinued olanzapine-fluoxetine combination (OFC) due to financial concerns. Four weeks later he became hypomanic, and despite starting valproate at that point, within 6 weeks he was hospitalized with a full manic episode. He was eventually re-started on OFC along with lithium and lamotrigine. Shortly afterward his clinical status improved markedly on all assessments by the end of the study.

At 9 months subject 2 had a discrete and severe stressful event (death of cousin, as mentioned earlier) that resulted in a rapid decompensation over a single week. To treat this relapse she received a course of electroconvulsive treatment (electroconvulsive treatment, 4 acute and 4 at weekly intervals), and subsequently showed improvement, but without achieving the level of sustained improvement that she had experienced earlier in the trial. Despite this stress-precipitated relapse at 9 months, earlier in the study she had shown resilience to stress when her cousin committed suicide at 2 months post-CKS. This loss had particular meaning for the subject as she and her cousin had been raised as sisters, and her cousin also struggled with intense and chronic suicidal ideation with a history of multiple suicide attempts and hospitalizations. The subject and her cousin had made a pact not to suicide, a significant barrier to a repeated suicide attempt in the 2 years prior to study entry. Her resilience in the context of such a significant loss was supported by her observation that she was grieving but she did not feel ‘depressed’ after the suicide. She did rate higher on the HDRS, CGI-S and lower on the CGI-I at the 2-month assessment, but then improved by the 3 and 6-month assessments.

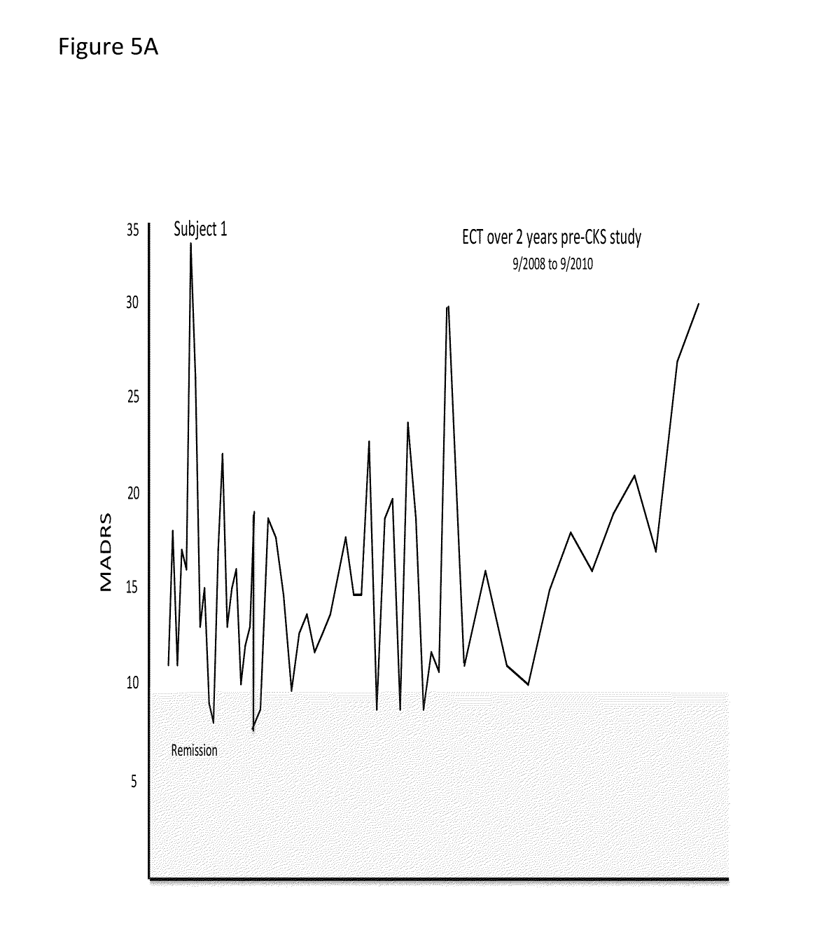

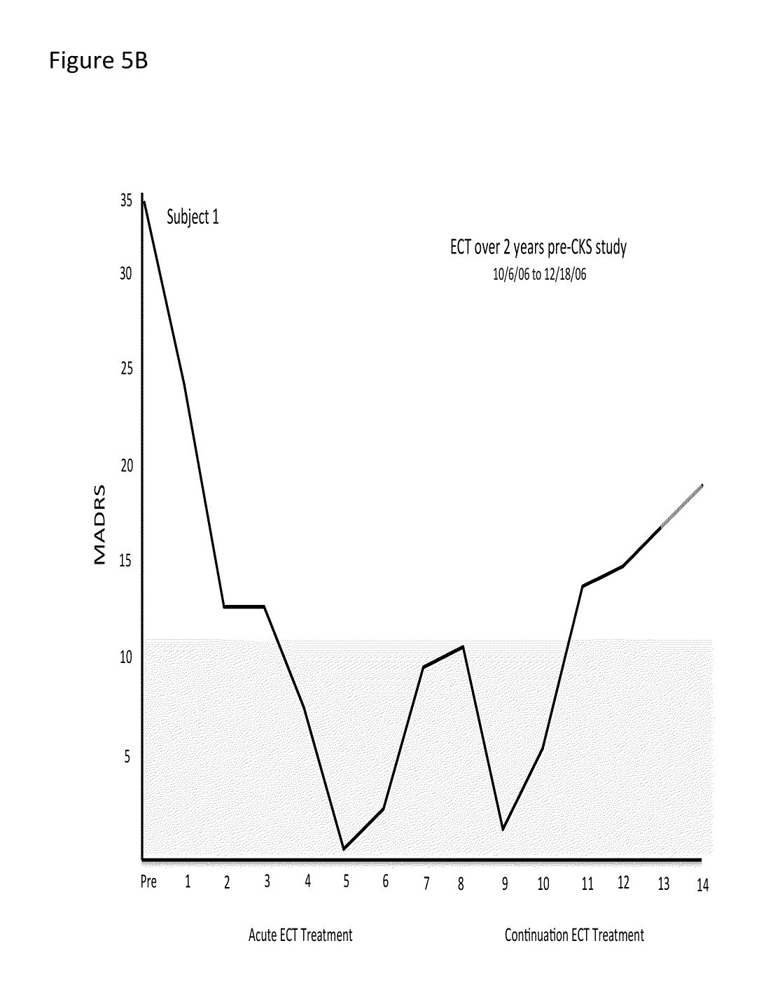

Both subject 1 and 2 had received ECT treatment prior to study entry and were assessed with the Montgomery-Asberg Depression Scale (MADRS) prior to each treatment (FIG. 5A and Figure B). Subject 1 had a course of 8 acute treatments, and 6 weekly continuation treatments (see FIG. 5B). The MADRS score fell into the remitted range within the first 6 treatments, but he then relapsed shortly afterward during continuation treatment. The decision to stop ECT rather than return to acute treatment after the relapse was due to the onset of paroxysmal atrial fibrillation that was difficult to control after the last 2 ECT procedures. A cardiology consult recommended against further ECT treatments, and it was discontinued.

Subject 2 had ECT over a 33-month period prior to the CKS procedure. The MADRS scale was used to assess the subject prior to each treatment (FIG. 5A) as well as the CGI-S and GAF scores done with each clinic visit. There was substantial variability in CGI-S scores from the years of 2002 to 2008 when the ECT treatments commenced. ECT effectively reduced the wide fluctuations in the scores, but without a decrease in symptom severity or improved function. ECT treatments continued despite the lack of full symptomatic recovery as she had a history of several near lethal suicide attempts as well as multiple hospitalizations. ECT appeared to reduced suicidality, as there were no suicide attempts or hospitalizations during the time she received ECT treatment.

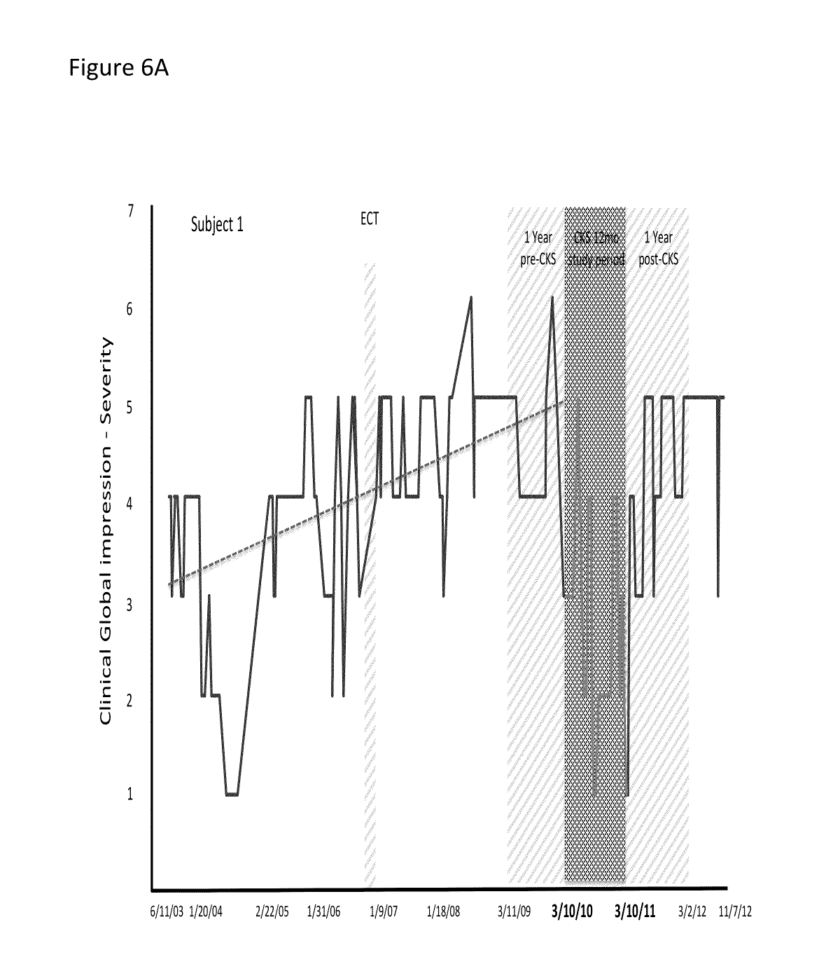

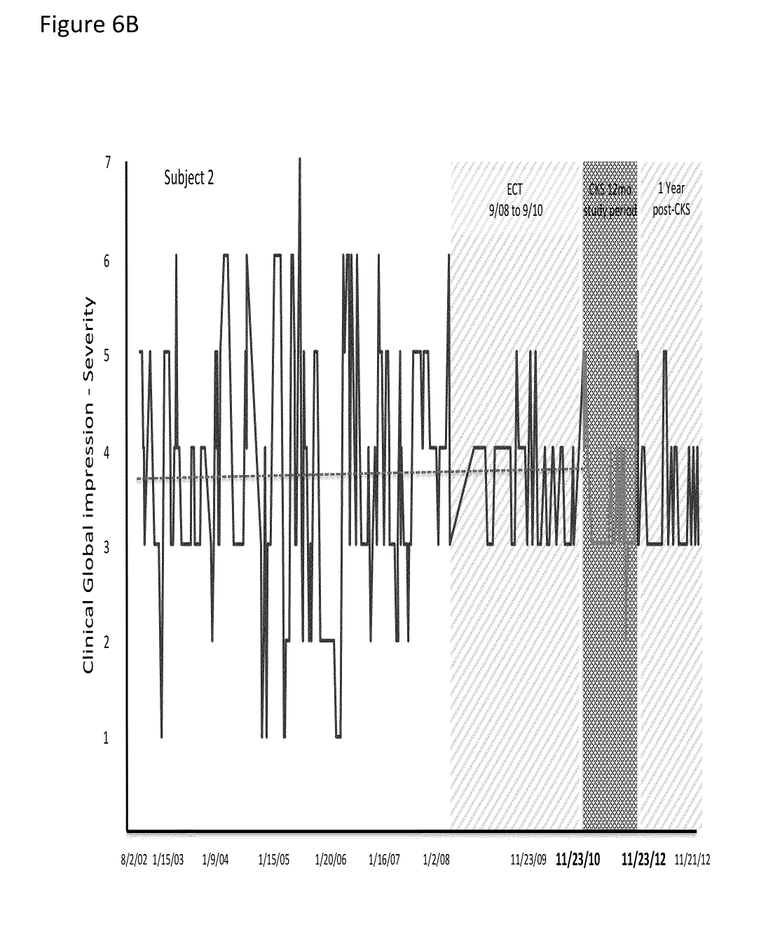

Longitudinal CGI-S scores for over a total of 10 years were available for subject 1 (201 assessments), 11 years for subject 2 (274 assessments) and are shown in FIGS. 6A and 6B. These assessments were made by a separate psychiatrist for each subject as part of the STEP-BD study and were done in the Bipolar Clinic. The dashed red line is a fitted trend for the years preceding the CKS study, and represents the trajectory of the illness course based on 6 or more years of assessments. The severe and refractory illness course prior to entry to the study is well illustrated. Subject 1 had a pattern of increasing severity of illness and decreasing function preceding the study, reflected in the upward trend in severity of depression and disability (FIG. 6A). Compared to the years prior to the procedure, the 12 months during the study showed a distinctly different course on the CGI-S, with improvement peaking at 6 months, then followed by a slow return to his severely depressed baseline which occurred about 4 months after study completion. Despite of depressive symptoms, the subject showed a consistent sustained higher level of function after the trial and to the present than at any point in the prior 7 years.

Subject 2 had CGI and Global Assessment of Functioning (GAF) assessments done in the clinic at regular intervals during continuation/maintenance ECT from April 2008 to September 2010 (a total of 119 treatments), see FIG. 6B. 9 months after the CKS procedure she had one course of 4 acute and 4 continuation treatments after the precipitated relapse described earlier. Since the study ended she has not required hospitalization nor further ECT treatments.

Subject 3 had a course of high frequency Transcranial Magnetic Stimulation (TMS) prior to study entry without benefit, yet after the CKS study was complete he had a course of right side low frequency TMS treatments (1 Hz, 10 minutes, 600 pulses, 120% motor threshold) and completely remitted over 5 weeks. With maintenance treatment he has remained relatively well, despite a long history of illness, and without a period of remission for more than 10 years.

Extensive serial MRI imaging of the brain did not reveal any visible structural change in either subject 1 or 2 at the target site or elsewhere. For reasons that remain unclear, there developed a bilateral area of necrosis in subject 3 that began to evolve at about 8 months post-procedure. The only known difference between this subject and the other 2 was a greater than 30 year history of alcohol dependence with intermittent cocaine abuse, though at the time of entry to the study he had been abstinent for 10 years.

Benefit of low-dose radiation targeted to the cingulate area 25. These described results for the treatment of refractory bipolar depression suggest that low-dose radiation targeted to the cingulate area 25 offers a non-invasive, yet highly precise and effective method to modulate neuronal activity in the brain. In terms of clinical status throughout the 12 month study and afterwards there was no worsening among the 3 subjects. Two subjects had a period of marked improvement up to the 6-month time point, and one sustained this improvement for 4 months. In the context of long term historical data on illness severity and disability by separate clinician ratings, the improvements noted in study assessments appears to represent a change from the expected course of illness.

Clinical outcome. The clinical outcome in terms of severity of symptoms, performance on serial neurocognitive tests, and functional capacity did not reveal any negative effects of treatment. Serial MRI volumetric imaging did not reveal any evidence of edema or necrosis in subject 1 or 2. In subject 3, MRIs at 9 months post procedure did show an area of edema at the target site that evolved to a necrotic lesion at 12 months. Despite a prior trial of adequate length of 10 Hz TMS prior to the study, subject 3 responded well to a course of right-side 1 Hz TMS immediately after the study.

Overall, subjects 1 and 2 had the best outcome during and after the trial. Both had events occur after the 6-month assessment that appeared to cause a worsening of depressive symptoms, but both began to rebound by 12 months. Subject 3 had little symptomatic improvement, but is now doing well for the first time in 10 years after a successful course of low frequency right-sided TMS. It is not clear that the CKS treatment had any influence on his subsequent response to TMS, but it is curious that he had not responded to an earlier adequate trial of high frequency TMS prior to the study.

Lesions. There was no evidence of a lesion in Cg25 by MRI at the target site in subject 1 and 2 at 12 months, however subject 3 did develop a lesion in this area. Radiation induced necrotic lesions have a long history of use in refractory neuropsychiatric illness for refractory obsessive compulsive disorder (OCD) and depression. However, for OCD, it has required >=120Gy to produce a lesion, in contrast to the 60Gy dose used in this study.

There are several reasons why Cg25 could be more radiosensitive than expected among cortical structures in subjects with bipolar disorder. The most obvious is the apparent hypermetabolism in this area with depression, and the atrophy of Cg25 seen in volumetric MRI and post-mortem studies. Animal studies have shown that the loss of glia is largely responsible for the atrophy observed in Cg25. The connection between hypermetabolism in Cg25 in the depressed state and possible glial apoptosis is not known, but persistently driven metabolic demand may slowly tip glia towards programmed cell death, and once triggered would be irreversible.

These findings suggest that RSN targeting Cg25 may be a useful tool for the treatment of severe refractory bipolar depression. There was no worsening of any subject's clinical status, no new impairment or disability, and no impairment detected by serial cognition assesments. Overall the procedure appears to have resulted in sustained clinical benefit for at least 2 of the 3 subjects.

TABLE 3
Subject demographics and characteristics

| Demographics | Subject 1 | Subject 2 | Subject 3 |
|---|---|---|---|
| Age at time of study entry | 60 | 35 | 65 |
| Signed consent | 4/21/10 | 9/27/10 | 7/6/2011 |
| Sex/race | M/White | F/Asian | M/White |
| Marital status | D | SO | S |
| Children | 3 | 0 | 0 |
| Disability | 2003 | 2001 | Part time |
| | | | contractor |
| Age first received any psychiatric care | 37 | 12 | 20 |
| Diagnoses prior to BP diagnosis | MDD, Conduct | MDD, Avoidant PD | Schizoid PD, |
| | Disorder MJ | Social Anxiety | ADD, Major |
| | abuse | Disorder | Depression, |
| | | | dysthymia |
| Substance abuse (last used) | Marijuana, | None | ETOH cocaine |
| | (6 mo prior to study) | | (10 yrs) |

Family history

| | | | |
|---|---|---|---|
| Bipolar disorder | None | 2 suspected: Mother, | 1 suspected: |
| | | maternal aunt | maternal aunt |
| Other Axis 1 diagnosis | Major depression: | Substance abuse | Maternal aunt |
| | mother, sister, | maternal cousin | post-partum |
| | maternal uncle | | depression |
| | | | maternal niece |
| | | | major depression |
| Completed suicide | 1 maternal uncle | 1 maternal cousin | No |
| ECT | None | None | Maternal aunt |

TABLE 4
Illness Course

| | Subject | | |
|---|---|---|---|
| | 1 | 2 | 3 |
| Followed in STEP-BD program since | 2003 | 2002 | NA |
| Age at onset | mid-30s | 16 | 15 |
| Age of diagnosis (BP1 or BP2) | 45 | 24 | 54 |
| Age of first hypomanic/manic episode | 42 | 16 | 20 |
| Date of onset for current MDE | 2005 (observed) | 2006 (observed) | 1997 (reported) |
| Duration of current MDE at study entry | 5 yrs | 5 yrs | 14 yrs |
| Longest period of observed | 10 mo 2004-5 | 5 mo 2006 | N/A |
| improvement: | | | |
| (CGI-S scores<=2, GAF>=60) | | | |
| Number of prior MDE | 3 chronic | >25 | >5 |
| | | Recur and chronic | Recur and chronic |
| History of a psychotic mood episode | With Mania | None | With MDE |
| History of rapid cycling | − | + | + |
| | | | (late 1990′s) |
| Last manic or hypomanic episode | 2004 | 2004 | 2009 |
| Number of prior hypomanic/manic | 3 manic: | >5 | >3 |
| episodes | 2001, 2003, 2004 | | |
| | 1 hypomania 2006 | | |
| Number of hypomania/manic episodes | >1 | >5 | >1 |
| while on medication | | | |
| Hypomania and/or mania during study | 1 manic episode | None | None |
| Number of past serious suicide attempts | 2 | 3 | None known |
| Number of past hospitalizations | 3 | >5 | 4 |

Abbreviations:
Recur recurrent,
MDE major depressive episode,
CGI-S clinical global impression-severity,
GAF global assessment of function,
mo months,
yr years,
AD antidepressant,
Y yes.
Date of onset of current MDE started after a 2 month period being well.

TABLE 5
Treatment history

| | | Subject | | |
|---|---|---|---|---|
| | | 1 | 2 | 3 |
| Treatment at study | Mood stabilizer | OLZ (OFC) | ZPN, clozapine | Lithium |
| entry | Antidepressant | FLX (OFC) | None | None |
| | | BPN, SRT | | |
| At end of study | Mood stabilizer | OLZ (OFC) | ZPN, clozapine | Lithium |
| | Antidepressant | FLX (OFC) | None | None |
| | ECT | 0 | 8 | 0 |
| Lifetime | Mood stabilizer | Lithium | Lithium VPA, | Lithium |
| | | VPA, LTG | CBZ, LTG | LTG, VPA |
| | | OLZ, APZ, ZPN | OLZ ARIP ZPN | QTP, RSP |
| | | RSP | QTP, RSP | Intoletrance APZ |
| | Antidepressant | TCN. tSLG, FLX, | PRX VEN FLX, | eCTP, CTP, FLX, |
| | | SRT BPN, DLX, | CTP, MRT, BPN, | BPN, NFD, DLX, |
| | | VEN, MDF | rMDF | >1 TCA |
| | Psychotherapy | LT: ST, PD | LT: ST, CBT BRF: | LT: ST, PD |
| | | BRF: CBT, DBT | CBT, DBT | |
| | Prior ECT courses | 1 | 1 (with 2 yrs maint) | 3* |
| | Total number of ECT | 14 | 56 | NK |
| | tx prior to study | | | |
| | Total number ECT | 0 | 8 | 0 |
| | during study | | | |
| | TMS 120% MT | 0 | 0 | 1 course of 25 |
| | 10 HZ left DLPFC | | | treatments |

Abbreviations:
CBT cognitive behavioral therapy,
IPT interpersonal therapy,
PD psychodynamic,
ST supportive,
DBT dialectical behavior therapy,
BRF brief,
LT long term.
VPA valproate,
CBZ carbazepine,
LTG lamotrigine,
OLZ olanzapine,
OFC olanzapine/fluoxetine combination,
ARP aripiprazole,
ZPN ziprasidone,
QTP quetiapine,
RSP risperidone,
FLX fluoxetine,
PXN paroxetine,
SRT sertraline,
CTP citalopram,
eCTP escitalopram,
VEN venlafaxine,
DLX duloxetine,
MRT mirtazapine BPN,
NFD nefazadone,
bupropion,
MDF modafanil,
rMDF armodafanil,
TCN tranylcypromine,
tSLG transdermal selegiline,
Tx treatments.
*The first acute/continuation ECT course was discontinued after 14 total treatments due to poor response and difficult to control paroxysmal atrial fibrillation with the last 2 ECT treatments. Two other acute courses were attempted in 2007 and 2009, but were discontinued after the first treatment due to return of atrial fibrillation.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### 10. REFERENCES

Alvarez J A & Emory E (2006). Executive Function and the Frontal Lobes: A Meta-Analytic Review. Neuropsych Rev 16(1):17-42.
Bush G et al. (2000). Cognitive and emotional influences in anterior cingulate cortex. Trends Cogn Sci 4(6):215-222.
Busner J & Targum S D (2007). The Clinical Global Impressions Scale. Applying a Research Tool in Clinical Practice. Psychiatry 4(7): 28-37.
Delis D C et al. (2000). California Verbal Learning Test, Second Edition (CVLT-II). Publisher: Pearson.
Drevets W C et al (2008). The Subgenual Anterior Cingulate Cortex in Mood Disorders. CNS Spectr. 13(8):663-681.
Folstein M F et al. (1975). Mini Mental State: a practical method for grading the cognitive state of patients for the clinician. J Psychiat Res 12:189-198.
Hamilton A (1960). A rating scale for depression. J Neurol Neurosurg Psychiatry 23:56-62.
Haskins B et al. (1995). Affect processing in chronically psychotic patients: development of a reliable assessment tool. Schizophrenia Research 15: 291-297.
Hoffelt S C (2006). Gamma Knife vs. CyberKnife. Oncology Issues September/October, pages 18-20.
Homack S et al. (2005). Test Review: Delis-Kaplan Executive Function System. J Clin Exp Neuropsychology 27(5): 599-609.
Lohoff F W (2010). Overview of the genetics of major depressive disorder. Curr Psychiatry Rep 12(6):539-546.
Mayberg HS et al. (2005). Deep Brain Stimulation For Treatment-Resistant Depression. Neuron 45:651-660.
Sole B et al. (2012). Neurocognitive impairment across the bipolar spectrum. CNS Neurosci Ther 18(3):194-200.
Wechsler D (1981). WAIS-R Manual. New York: Psychological Corporation.
Yatham L N et al. (2010). The International Society for Bipolar Disorders-Battery for Assessment of Neurocognition (ISBD-BANC). Bipolar Disorders 12(4):351-363.
Young R C et al. (1978). A rating scale for mania: reliability, validity and sensitivity. Brit J Psych 133:429-435.

What is claimed is:

1. A method of treating a subject suffering from a neuropsychiatric disorder, the method comprising delivering a therapeutically effective dose of radiation to an area of said subject's subgenual anterior cingulate cortex, wherein said radiation does not exceed 75 Gray in said area and wherein said delivering of radiation is effective to achieve an improvement of said neuropsychiatric disorder, as measured using standard protocols for assessing improvement.

2. The method according to claim 1, wherein said neuropsychiatric disorder is depression, major depressive disorder, bipolar depression, schizophrenia, autism or an anxiety disorder.

3. The method according to any of claim 1 or 2, wherein said neuropsychiatric disorder is refractory to treatment.

4. The method according to any of claim 1, 2 or 3, wherein said area is the cingulate area 25.

5. The method according to any of claims 1-4, wherein said radiation is delivered at least once daily for at least two consecutive days.

6. The method according to any of claims 1-5, wherein said radiation is administered at a dose of 10-30 Gray.

7. The method according to any of claims 1-5, wherein said radiation is administered at a dose of 30-60 Gray.

8. The method according to any of claims 1-5, wherein said radiation is administered at a dose of 60-75 Gray.

9. A method for improving neurocognitive function in a subject, the method comprising delivering a therapeutically effective dose of radiation to an area of said subject's subgenual anterior cingulate cortex, wherein said radiation does not exceed 75 Gray in said area and wherein said delivering of radiation is effective to achieve improvement in neurocognitive function, as measured using standard protocols for assessing neurocognitive function.

10. The method according to claim 9, wherein said area is the cingulate area 25.

11. The method according to any of claim 9 or 10, wherein said radiation is delivered at least once daily for at least two consecutive days.

12. The method according to any of claims 9-11, wherein said radiation is administered at a dose of 10-30 Gray.

13. The method according to any of claims 9-11, wherein said radiation is administered at a dose of 30-60 Gray.

14. The method according to any of claims 9-11, wherein said radiation is administered at a dose of 60-75 Gray.

15. The method according to claim 9, wherein the subject has a neuropsychiatric disorder that impairs neurocognitive function.

16. The method according to claim 15, wherein said neuropsychiatric disorder is refractory to treatment.

17. The method according to any of claim 15 or 16, wherein the subject has depression, major depressive disorder, bipolar depression, schizophrenia, autism or an anxiety disorder.
